(19) United States
(12) Patent Application Publication
(10) Pub. No.: US 2026/0347433 A1
(43) Pub. Date: Nov. 5, 2026

(54) NATIVE COLLAGEN EXTRACTION FROM FISH SCALES

(71) Applicants: HI5, Bayonne (FR); Centre National de la Recherche Scientifique, Paris (FR); Université de Pau et des Pays de I'Adour, Pau (FR)

(72) Inventors: Arnaud PETITPAS, Paris (FR); Erik DE LAURENS, Paris (FR); Judith Viviane Renée VEILLON, Corbeil-Essonnes (FR); Susana Cristina DE MATOS FERNANDES, Pau (FR)

(21) Appl. No.: 19/488,910

(22) PCT Filed: May 31, 2024
(86) PCT No.: PCT/EP2024/065121

§ 371 Date: Dec. 1, 2025

(30) Foreign Application Priority Data
Jun. 2, 2023 (FR) FR2305559

(51) Int. Cl. C07K 14/78; C07K 1/14

(52) U.S. Cl. CPC C07K 14/78; C07K 1/145

(57) ABSTRACT

A device for the mechanical extraction of native collagen obtained from dried fish scales, including a tank designed to receive a batch of fish scales, a member for closing the tank positioned at the top of the tank, motorized rotating blades arranged in the tank and designed to defibrate the fish scales, and elements for mechanically separating the native collagen fibers from other parts of the fish scales resulting from defibration.

### FIELD OF THE INVENTION

The invention relates to the extraction of native collagen obtained from fish scales.

### BACKGROUND

The fishing and aquaculture industries produce large quantities of waste, including fish scales which are one of the least valorized by-products. Several hundred thousand metric tons of fish scales are in fact generated every year by the fishing and aquaculture industries. Currently, the fish scales are by-products which are valorized only to a very limited extent. However, the collagen and the hydroxyapatite which form the scale are molecules of interest with high added value.

Collagen is the most widespread biopolymer in the animal world. It is a polypeptide chain composed of amino acids, present in particular in the skin and the bones. Skin and bones of porcine, ovine and marine origin are also the most common sources for procuring collagen and its hydrolyzed form, gelatin. These components are very useful for the agribusiness industry, where they are used as binder and thickener. The cosmetics and nutraceutical sectors are also important markets, especially in view of the biocompatible nature of collagen. This is equally true of the biomedical industry, which is facing an increased demand for native collagen, although at a lower scale. Hydroxyapatite is a biomineral that is also present in the mineralized structures of the animal world. It has interesting properties, especially for the nutraceutical, biomedical and cosmetics sectors.

Fish scales are still rarely used for the extraction of native collagen, fish skins being preferred. The standard method used to extract collagen from fish scales is based directly on the methods used to extract collagen from skins. Currently therefore, the collagen obtained from fish scales is mainly extracted by chemical process with the use of acidic and basic solutions to break down the natural material and isolate the targeted molecules.

The standard extraction technique consists of pretreatment with a highly basic solution such as caustic soda, followed by treatment based on acetic or hydrochloric acid. EDTA (ethylenediaminetetraacetic acid), a chelating agent that complexes heavy metals, is often used to demineralize the scales before the extraction step. The standard steps for the extraction of native collagen in the laboratory are as follows:
pretreatment with caustic soda,
demineralization of the scales with EDTA,
extraction of the collagen at 4 °C using acetic acid,
centrifuging,
salting out, to purify the collagen,
dialysis, and
freeze drying.

A step of enzyme hydrolysis can be added to, or replace the acetic acid extraction phase to improve the yield.

This extraction method presents several disadvantages. Firstly, it does not follow the precepts of green chemistry with the use of numerous chemical agents. Also, the various hydrolyses are difficult to control, which may lead to a risk of denaturing the molecules of interest extracted. The collagen chains are broken randomly, therefore making the nature of the peptides produced difficult to predict and to control.

In addition, this extraction may take several days and have a limited yield. Lastly, it consumes a large quantity of water and the use of a freeze dryer at end of extraction consumes a large quantity of energy (of the order of 1,500 to 2,500 kWh per metric ton of water evaporated). Extraction is therefore expensive and energy-consuming.

### SUMMARY

The invention therefore aims to provide a device for the extraction of native collagen obtained from fish scales which is faster and less energy-greedy than the methods of the prior art. The invention can also be used to obtain a yield that is greater than that of the prior art and that has a higher degree of purity. Lastly, the invention can be used to obtain native collagen fibers retaining a natural structure.

The invention therefore relates to a device for the mechanical extraction of native collagen obtained from dried fish scales comprising:
a tank designed to receive a batch of fish scales,
a member for closing the tank positioned at the top of the tank,
motorized rotating blades arranged in the tank and designed to defibrate the fish scales, and
means for mechanically separating the native collagen fibers from other parts of the fish scales resulting from defibration.

Thus, the extraction device does not involve the use of chemical products or the need to solubilize the collagen, which avoids denaturing the collagen fibers extracted. Such an extraction, over a period ranging from 10 seconds to 3 minutes, can be used to obtain an optimum degree of purity of native collagen fibers having an impurity content ranging from 0.3% to 20%. Lastly, the fact that extraction is carried out entirely mechanically considerably reduces the consumption of water and energy.

According to other optional characteristics of the extraction device taken alone or in combination:
the extraction device comprises means for cleaning and drying the fish scales before they are placed in the tank. The means for preparing the fish scales are therefore included in the device;
the means for cleaning the fish scales comprise at least a fresh water bath;
the means for drying the fish scales comprise at least one support grid and at least one hot air blower;
the drying means can be powered by a renewable energy source and/or a fatal energy source.
the tank is made of an alloy of hardness greater than that of hydroxyapatite. This limits the pollution of native collagen fibers by steel particles;
the tank is made at least partly of stainless steel;
the tank is arranged on a shaft so that it can be tilted. The tank is therefore easier to empty;
the extraction device comprises means for adjusting the height of the closure member on the tank. The tank volume can therefore be varied by adjusting the position of the closure member;
the means for adjusting the height of the closure member on the tank comprise adjustment screws and/or notched adjustment elements;
the motorized blades are mounted on a shaft that is connected to a motor;
at least one motorized blade consists of a metal bar, for example of straight rectangular cross-section, comprising a central perforated portion connected to the shaft and at least one terminal portion forming an angle of between 30° and 70° with the central portion;
at least one motorized blade consists of a metal bar, for example of straight rectangular flat cross-section; the motor is designed to rotate the motorized blades at a speed of between 5,000 and 30,000 rpm;
the motor is coupled to a variable frequency drive. The speed of rotation of the motorized blades can therefore be varied depending on the type and quantity of scales present in the tank;
the shaft is threaded. The motorized blades can therefore be easily attached to the shaft;
the shaft is made of stainless steel;
at least one spacer is arranged on the shaft between two successive motorized blades;
the means for mechanically separating the native collagen fibers from other parts of the fish scales resulting from defibration comprise at least one system for sucking the content of the tank placed opposite an opening of the tank, the tank comprising means arranged to prevent the passage of native collagen and allow the passage of hydroxyapatite and impurities. The native collagen can therefore be separated from other products resulting from defibration at the same time as said defibration of the fish scales;
the extraction device comprises a member for measuring the temperature inside the tank. Its purpose is to detect possible overheating inside the tank which could denature the collagen fibers;
the member for measuring the temperature inside the tank is coupled to a member for lowering the temperature inside the tank. Its purpose is to avoid possible overheating inside the tank which could denature the collagen fibers;
the member for lowering the temperature inside the tank comprises at least one element selected from: a system for stopping the motorized blades, a system for limiting the speed of rotation of the motorized blades or a device for injecting at least one cooling agent, for example liquid nitrogen.

The invention also relates to a method for the mechanical extraction of native collagen obtained from fish scales using an extraction device according to the invention, the steps of the method being as follows:
cleaning and drying the scales,
inserting scales in the tank,
mechanically defibrating the scales by rotating the motorized blades, and mechanically separating the native collagen fibers from other parts of the fish scales resulting from defibration.

According to other optional characteristics of the extraction method taken alone or in combination:
cleaning is selected from cleaning with water or enzyme cleaning;
the height of the closure member above the tank is selected according to the volume of fish scales in the tank;
the tank is inclined during the step of mechanically defibrating the scales by rotating the motorized blades;
the motorized blades are rotated at a speed of between 5,000 and 30,000 rpm, preferably between 15,000 and 25,000 rpm;
the motorized blades are rotated for a period of between 10 seconds and 3 minutes;
the speed of rotation of the motorized blades varies during the step of mechanically defibrating the scales by rotating the motorized blades;
a suction system sucks the content of the tank during the step of mechanically defibrating the scales by rotating the blades, the tank comprising means preventing the native collagen fibers from coming out and allowing the hydroxyapatite and impurities to come out;
the temperature inside the tank is measured during the step of mechanically defibrating the scales by rotating the motorized blades;
the temperature inside the tank is lowered during the step of mechanically defibrating the scales by rotating the motorized blades; and
the temperature inside the tank is lowered during the step of mechanically defibrating the scales by rotating the motorized blades by reducing the speed of rotation of the motorized blades, by stopping the rotation of the motorized blades, or by injecting at least one cooling agent, for example liquid nitrogen.

### Brief Description of the Figures

The invention will be better understood on reading the following description, given solely by way of example and with reference to the accompanying drawings in which:

FIG. 1 is a cross-sectional view of a device for the mechanical extraction of native collagen obtained from fish scales according to one embodiment of the invention, and

FIG. 2 is a diagrammatic representation of a method for the mechanical extraction of native collagen obtained from fish scales according to one embodiment of the invention.

### DETAILED DESCRIPTION

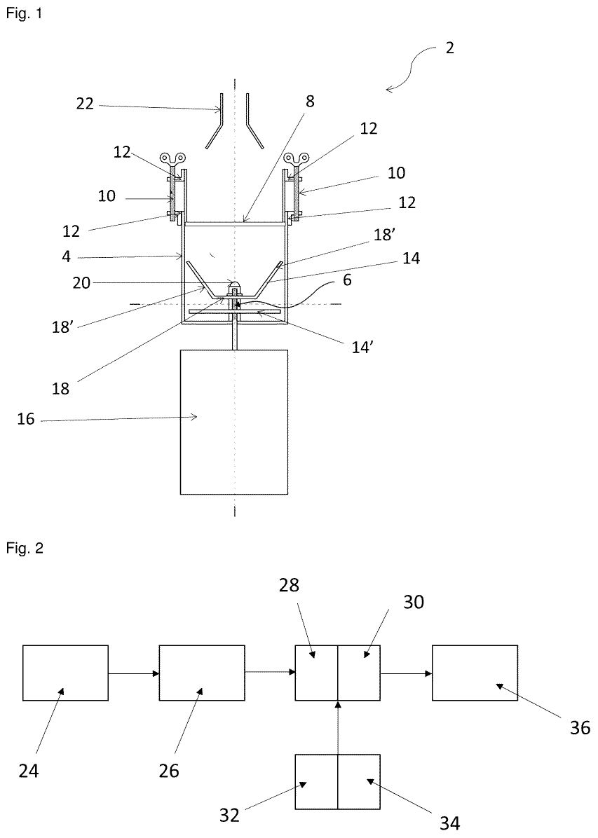

We now refer to FIG. 1 showing a device 2 for the mechanical extraction of native collagen obtained from dried fish scales. “Native collagen” designates a non-denatured collagen that has retained its triple helix structure. “Mechanical extraction” designates an extraction of native collagen not requiring the use of chemicals such as those mentioned previously, for example by successive baths.

Traditionally, the fish scales are washed then dried before the phase of mechanically extracting native collagen. To do this, the extraction device 2 comprises means for cleaning and drying the fish scales before mechanically extracting the native collagen. Alternatively, the extraction device can receive fish scales that have already been washed and dried.

The means used to clean the fish scales may comprise at least one fresh water bath, preferably slightly acidic, to rinse the fish scales. It may also consist in cleaning by enzyme washing.

The means used to dry the fish scales may include at least one support grid and at least one hot air blower. Advantageously, the drying means are powered by a renewable energy source and/or a fatal energy source.

The extraction device 2 comprises a tank 4 designed to receive the dried fish scales. The tank comprises at least one opening for introducing fish scales.

The tank 4 is preferably made of an alloy of hardness greater than that of hydroxyapatite. This limits the pollution of native collagen fibers by steel particles. For example, the tank 4 can be made at least partly of stainless steel (which also makes the tank 4 easier to clean).

The tank 4 is arranged on a shaft 6 so that it can be tilted. This possibility of tilting makes it easier to empty the tank 4. In addition, inclining the tank 4 also allows the native collagen to be separated from the other components obtained from the fish scales. To allow such tilting, the tank 4 is positioned on a tilting shaft, facilitating the separation of the phases by gravity during the process.

The volume of the tank 4 can be adjusted, in particular according to the volume of fish scales placed in the tank 4. It is in fact preferred to limit the movement of the fish scales in the tank 4 to facilitate defibration by shearing the fish scales and therefore the extraction of native collagen. To do this, means for adjusting the height of the closure member 8 on the tank 4 can be provided. These adjustment means can consist of adjustment screws 10, as shown on FIG. 1. The closure member can therefore have a U-shaped cross-section (for example a cylinder having a wall at one of its ends if the tank has a circular opening). Each adjustment screw 10 cooperates with two supports 12 crossed by threaded holes, one carried by the tank 4 and the other by the closure member 8. The relative screwing of the adjustment screws 10 on the supports 12 carried by the closure member 8 with respect to that of the adjustment screws 10 on the supports carried by the tank 4 is used to adjust the height of the member 8 for closing the tank 4 on the latter. Instead of using adjustment screws 10, a notched adjustment element can be used, for example by means of pairs of notched rods placed respectively on the tank 4 and on the closure member 8.

The extraction device 2 also comprises motorized rotating blades 14 and 14′, arranged in the tank 4 and designed to defibrate the fish scales. The blades act as flails when they are rotated, thereby defibrating the fish scales present in the tank 4.

The motorized blades 14 and 14′ are rotated by the shaft 6, connected directly or indirectly to a motor 16. The motorized blades 14 and 14′ are mounted removably on the shaft 6. The extraction device 2 is therefore easy to maintain and modular since motorized blades of a given shape can be replaced by motorized blades of another shape. As an alternative, the motorized blades could be part of a rotating unit made in one piece.

As shown on FIG. 1, at least one motorized blade, in this case the motorized blade 14, can consist of a metal bar, for example of straight rectangular cross-section, comprising a central portion 18 that is preferably perforated (i.e. comprising a through-hole) connected to the shaft 6 and at least one terminal portion, in this case two terminal portions 18′, forming an angle of between 30° and 70° with the central portion (the angle is equal to about 45° on FIG. 1). As an alternative or in addition, at least one motorized blade, in this case the motorized blade 14′, can be flat and preferably also perforated.

Advantageously, the shape of at least one part of the motorized blades prevents the fish scales from stagnating in the tank 4. In the example shown, the flat motorized blade 14′ is placed at the bottom of the tank 4 to avoid such stagnation.

As explained above, the shaft 6 is connected to a motor 16. This motor 16 can be designed to rotate the motorized blades 14 and 14′ at a speed of between 5,000 and 30,000 rpm, preferably between 15,000 and 25,000 rpm, this range of speeds being the most suitable. Generally, the speed of rotation of the motorized blades 14 and 14′ can be varied depending on the type and quantity of scales present in the tank 4.

Advantageously, the motor is coupled to a variable frequency drive. This allows the speed of rotation to be adjusted from one defibration step to another for different batches of scales, or to vary the speed of rotation of the motorized blades 14 and 14′ during a given defibration step if this is advantageous.

The shaft 6 is preferably made of stainless steel for the same reasons as for the tank 4. It is preferably threaded, as shown on FIG. 1. This may allow the motorized blades 14 and 14′ to be screwed if the through-hole of the motorized blades 14 and 14′ is threaded. In this case, clamping can be carried out using a clamping bolt 20.

At least one spacer can be placed between the successive motorized blades 14 and 14′. This allows the distance separating the motorized blades 14 and 14′ to be adjusted and thus adapted to the type and volume of scales present in the tank 4.

The extraction device further comprises, and as explained above, a member 8 for closing the tank 4 placed above the tank 4. It could be a solid plate for example.

The extraction device 2 further comprises means for mechanically separating the native collagen fibers from other parts of the fish scales resulting from defibration.

In the example shown on FIG. 1, these mechanical separation means comprise at least one suction system 22 placed opposite an opening through which fish scales are placed in the tank 4. This suction system 22 is designed and placed so as to suck the content of the tank 4. In this case, the tank 4 comprises means arranged to prevent the passage of native collagen and allow the passage of hydroxyapatite and impurities. The tank 4 may for example comprise openings in order to extract from the tank 4 certain products resulting from defibration of the fish scales, while preventing other products resulting from this defibration from coming out of the tank 4. The other components resulting from defibration such as hydroxyapatite are removed from the tank 4 at the same time as defibration is carried out by the motorized blades 14, so that only the purest possible native collagen is left in the tank 4.

Advantageously, the extraction device 2 may comprise a member for measuring the temperature inside the tank 4, for example a temperature sensor arranged in the tank 4. Its purpose is to detect possible overheating inside the tank 4 which could denature the collagen fibers. To avoid any risk of denaturing the native collagen, in fact, the temperature inside the tank should preferably be kept below 60 °C.

With the same objective of controlling the temperature, the member for measuring the temperature inside the tank 4 can be coupled to a member for lowering the temperature inside the tank 4. Its purpose is to avoid possible overheating inside the tank which could denature the collagen fibers. Advantageously, the member for lowering the temperature inside the tank 4 comprises at least one element selected from: a system for stopping the rotation of the motorized blades 14 and 14′, a system for limiting the speed of rotation of the motorized blades 14 and 14′, or a device for injecting at least one cooling agent, for example liquid nitrogen, for example all around the tank.

Concerning the method for the mechanical extraction of native collagen obtained from fish scales using an extraction device according to the invention, the first step 24 may consist in cleaning (with fresh water or enzyme cleaning) and drying the fish scales using the means described above.

After this step, a volume of fish scales is placed inside a tank 4 (step 26). The height of the closure member 8 on the tank 4 can then be adjusted as explained above, in particular according to the volume of fish scales placed in the tank 4.

A step 28 of defibrating the fish scales by rotating the motorized blades 14 and 14′ is then implemented. As explained above, the motorized blades 14 and 14′ can be rotated at a speed of between 5,000 and 30,000 rpm, preferably between 15,000 and 25,000 rpm, for a period varying preferably from 10 seconds to 3 minutes. The speed of rotation of the motorized blades may vary during the step 28 of defibrating the fish scales.

During this step 28 of defibrating the fish scales, the tank 4 can be inclined in order to mechanically separate the native collagen fibers from other elements resulting from defibration.

As explained above and as shown on FIG. 2, the step 30 of mechanically separating the native collagen fibers from other elements resulting from defibration can be carried at the same time as that of defibrating the fish scales. It can be carried out by the suction system 22 described above. As an alternative, the collagen can be mechanically separated from the other elements resulting from defibration after defibration, for example by recovering the defibrated scales then passing them over a vibrating sieve.

A step 32 of measuring the temperature inside the tank 4 can be provided, for the reasons mentioned above, during the step of defibrating the fish scales. If the temperature inside the tank 4 increases above a threshold, a step 34 of lowering the temperature inside the tank 4 can be implemented by reducing the speed of rotation of the motorized blades 14 and 14′, by stopping the rotation of the motorized blades 14 and 14′, or by injecting at least one cooling agent such as liquid nitrogen.

Lastly, a step 36 of micronizing the native collagen fibers extracted, for example in a rotor mill, can be provided to increase the solubility of the collagen in the acid.

According to one embodiment of the invention, 20 grams of dry scales (residual moisture content about 11%) of tilapia (Oreochromis niloticus) are placed in the stainless steel tank 4 of diameter 14 cm. Two flat and straight stainless steel motorized blades 14′, of length 13.5 cm, and one motorized blade 14 of the shape described above (of length 6 cm and height 2 cm) are positioned one after the other on the shaft 6, the assembly being clamped and fastened together by a clamping bolt 20.

With the adjustment screws 10, a closure member 8 is positioned so that the height of the chamber defined by the tank 4 and the closure member 8 is 7 cm.

A suction system 22 is installed on the tank 4 to separate the products resulting from defibration. The tank 4 is provided with an air inlet to avoid creating a vacuum. The speed of the motorized blades is set at 28,000 rpm, and the step 28 of defibrating the fish scales lasts for 1 minute and 30 seconds. After defibrating the fish scales and mechanically separating the native collagen fibers from other elements resulting from defibration, 6 grams of pure native collagen (the impurity content is less than 2%) in fibrous form are collected.

The physico-chemical analyses performed demonstrate that the properties of this collagen are those of a native collagen having retained its triple helix structure. This collagen can then be solubilized in an organic acid, or inorganic acid (traditionally acetic acid), or used directly for the formation of biofilms or biomaterials, for example. A suction bag of the suction system 22 contains 14 grams of residue, mainly composed of mineral, with a hydroxyapatite content greater than 60%. The operation can then be repeated instantaneously by reloading the tank 4 with fish scales.

### LIST OF REFERENCES

2: extraction device
4: tank
6: shaft
8: closure member
10: adjustment screw
12: support
14, 14′: rotating blades
16: motor
18: central portion 18′: terminal portions
20: clamping bolt
22: suction system
24: cleaning and drying the fish scales
26: inserting the fish scales in the tank
28: defibrating the fish scales mechanically separating the native collagen fibers from other elements resulting from defibration
32: measuring the temperature inside the tank
34: lowering the temperature inside the tank
36: micronizing the native collagen fibers

1. A device for the mechanical extraction of native collagen obtained from dried fish scales comprising:
a tank designed to receive a batch of fish scales,
a member for closing the tank positioned at a top of the tank,
motorized rotating blades arranged in the tank and designed to defibrate the fish scales, and
means for mechanically separating native collagen fibers from other parts of the fish scales resulting from defibration.

2. The device according to claim 1, comprising means for cleaning and drying the fish scales before they are placed in the tank.

3. The device according to claim 1, wherein the tank is made of an alloy of hardness greater than that of hydroxyapatite.

4. The device according to claim 1, comprising means for adjusting a height of a closure member on the tank.

5. The device according to claim 1, comprising a shaft carrying the motorized blades and connected to a motor coupled to a variable frequency drive.

6. The device according to claim 1, wherein the means for mechanically separating the native collagen fibers from other parts of the fish scales resulting from defibration comprise at least one system for sucking the content of the tank placed opposite an opening of the tank, the tank comprising means arranged to prevent the passage of native collagen and allow the passage of hydroxyapatite and impurities.

7. The device according to claim 1, comprising a member for measuring a temperature inside the tank.

8. The device according to claim 7, wherein the member for measuring the temperature inside the tank is coupled to a member for lowering the temperature inside the tank.

9. A method for the mechanical extraction of native collagen obtained from fish scales using an extraction device according to claim 1, the method comprising steps of:
cleaning and drying the fish scales,
inserting the fish scales in the tank,
mechanically defibrating the fish scales by rotating the blades, and
mechanically separating the native collagen fibers from other parts of the fish scales resulting from defibration.

10. The method according to claim 9, wherein a height of a closure member above the tank is selected according to a volume of the fish scales in the tank.

11. The method according to claim 9, wherein a suction system sucks content of the tank during the step of mechanically defibrating the fish scales by rotating the motorized blades, the tank comprising means preventing the native collagen fibers from coming out and allowing hydroxyapatite and impurities to come out.

12. The method according to claim 9, wherein a temperature inside the tank is measured during the step of mechanically defibrating the fish scales by rotating the motorized blades.

13. The method according to claim 12, wherein the temperature inside the tank is lowered during the step of mechanically defibrating the fish scales by rotating the blades.
